(19) United States
(12) Patent Application Publication
(10) Pub. No.: US 2015/0057657 A1
(43) Pub. Date: Feb. 26, 2015

(54) MEDICAL BALLOON HAVING PATTERNED RECESSED WALL PROFILE

(71) Applicant: Boston Scientific Scimed, Inc., Maple Grove, MN (US)

(72) Inventors: Robert N. Squire, Maple Grove, MN (US); Jeffrey S. Lindquist, Maple Grove, MN (US); Derek C. Sutermeister, Ham Lake, MN (US); Patrick A. Haverkost, Brooklyn Center, MN (US); Timothy A. Ostroot, Cokato, MN (US); Martin R. Willard, Burnsville, MN (US); Daniel J. Horn, Shoreview, MN (US); Adam J. Royer, Brooklyn Park, MN (US); Ken Fredrikson, Howard Lake, MN (US)

(21) Appl. No.: 14/465,643

(22) Filed: Aug. 21, 2014

Related U.S. Application Data
(60) Provisional application No. 61/868,859, filed on Aug. 22, 2013.

(51) Int. Cl. A61B 18/14; B29D 23/00; B29C 49/20

(52) U.S. Cl. CPC A61B 18/1492; B29C 49/20; B29D 23/00; A61B 2017/00526
USPC 606/41; 264/512; 425/127

(57) ABSTRACT

A medical balloon comprising a balloon wall formed from a polymeric material, the balloon wall having an inner surface and an outer surface, the balloon wall comprising patterned recesses in the outer surface thereof and flexible circuits disposed within the patterned recesses, the flexible circuits are defined by an outer perimeter, and devices and methods for making the same.

### CROSS-REFERENCE TO RELATED APPLICATIONS

This application claims priority under 35 U.S.C. §119 to U.S. Provisional Application Ser. No. 61/868,859, filed Aug. 22, 2013, the entirety of which is incorporated herein by reference.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

In one aspect, the present disclosure relates to a medical balloon comprising a balloon wall formed from a polymeric material, the balloon wall having an inner surface and an outer surface, the balloon wall comprising patterned recesses in the outer surface thereof, and flexible circuits disposed within the patterned recesses, the flexible circuits are defined by an outer perimeter.

In another aspect, the present disclosure relates to a method of forming a medical balloon defined by a shape, the medical balloon having flexible circuits defined by an outer perimeter disposed on an outer surface of the balloon, the method including providing a balloon mold in the shape of the balloon, the balloon mold comprising a first inner surface diameter, providing an inner sleeve within the balloon mold that defines the perimeter of the flexible circuits, the inner sleeve comprising a second inner surface diameter that is less than the diameter of the first inner surface, providing a balloon preform, and radially expanding the balloon preform in the balloon mold to form a medical balloon, wherein the inner sleeve defines patterned recesses in the outer surface of the medical balloon.

In another aspect, the present disclosure relates to a mold for forming an expandable medical balloon configured to accept flexible circuits on an outer surface of the balloon, the flexible circuits defined by a perimeter, the mold including an outer shell defining the shape of the balloon, the outer shell comprising a body portion, and waist and cone portions, the body portion of the outer shell defined by a length and having an inner surface comprising a first diameter and an inner sleeve defining the perimeter of the flexible circuits, the inner sleeve having an inner surface comprising a second diameter, the second diameter is less than the first, the inner sleeve extends no further than the length of the body portion of the mold.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a side view of a catheter having a renal nerve modulation balloon according to the disclosure disposed on the distal end thereof.

FIG. 2 is a radial cross-section taken at section 2-2 in FIG. 1.

FIG. 3 is a radial cross-section taken at section 3-3 in FIG. 1.

FIG. 4 is a partial side view of a balloon as molded illustrating the patterned recesses in the outer surface of the balloon wall.

FIG. 5 is a perspective view of a balloon similar to that shown in FIG. 4 in an expanded state illustrating the patterned recesses in the outer surface of the balloon wall.

FIG. 6 is an expanded partial view of a balloon similar to that shown in FIGS. 4 and 5 in an expanded state illustrating the patterned recesses in the outer surface of the balloon wall.

FIG. 7 is a top down view of a flexible circuit for use on in combination with balloons as shown in FIGS. 4-5.

FIG. 8 is a perspective view of the flexible circuit similar to that shown in FIG. 7 embedded in the patterned recesses of a balloon similar to that shown in FIGS. 5 and 6.

FIG. 9 is a side view of an embodiment of a renal nerve modulation balloon having two flexible circuits disposed within patterned recesses in the balloon wall.

FIG. 10 is a side view of another embodiment of a renal nerve modulation balloon having four flexible circuits disposed within patterned recesses in the balloon wall.

FIG. 11 illustrates an embodiment of sleeve for use in a balloon mold for forming patterned recesses in the outer surface of the balloon.

FIG. 12A is an exploded view of a sleeve and a balloon mold for forming a balloon having patterned recesses in the outer surface of the balloon.

FIGS. 12B and 12C illustrate a sleeve partially inserted into a balloon mold.

FIG. 12D illustrates a sleeve fully inserted within a balloon mold.

FIG. 13 is a cross-sectional view of a portion of an example balloon and flexible circuit.

FIG. 14 is a side view of a portion of an example balloon.

FIG. 15 is a cross-sectional view of a portion of an example balloon and flexible circuit.

FIG. 16 is a cross-sectional view of a portion of an example balloon and flexible circuit.

### DETAILED DESCRIPTION

While embodiments of the present disclosure may take many forms, there are described in detail herein specific embodiments of the present disclosure. This description is an exemplification of the principles of the present disclosure and is not intended to limit the disclosure to the particular embodiments illustrated.

The present disclosure is directed to devices for percutaneous renal artery denervation, particularly expandable balloons and to methods of making and using the same.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal artery denervation can reduce blood pressure, and may be a viable treatment option for many patients with hypertension who do not respond to conventional drugs.

Ultrasound, radiofrequency energy, microwave energy, direct heating elements, and balloons with heat or energy sources may be applied to a region of sympathetic nerves.

A specific method for treatment of the renal sympathetic nerves involves a percutaneous, catheter-based therapy that uses radiofrequency energy to disrupt the renal sympathetic nerves. This method involves the use of an expandable medical balloon which is advanced to the treatment site, expanded, and energy is transmitted through the balloon via flexible circuits disposed on the outside of the balloon.

The flexible circuits are bonded to the outside surface of the renal denervation balloon.

Delamination caused by edge-lift of the flexible circuits and tear related issues can occur during balloon insertion, refolding and withdrawal. There remains a need in the art for improved balloons for renal artery denervation having high robustness.

The present disclosure relates to balloon for renal nerve modulation comprising a balloon wall having an interior and an exterior surface and flex circuits adhesively bonded to the exterior of a balloon. Renal nerve modulation or renal denervation is sometimes used to treat conditions relating to hypertension and/or congestive heart failure.

While the devices and methods disclosed herein are discussed relative to renal nerve modulation, it is contemplated that these devices and methods may be employed in other treatments as well.

The devices and methods according to the disclosure involve the delivery of radiofrequency energy to the renal nerve to temporarily or permanently modify nerve function.

Treatment involves delivery of the balloon to a treatment site via a catheter delivery device, inflation of the balloon at the treatment site, delivery of energy to the flexible circuit for nerve denervation, deflation and refolding of the balloon, and pulling of the balloon back into the catheter delivery device for withdrawal from the patient.

In alternative embodiments, other sources of energy such as ultrasound energy, microwave energy or direct heating elements may be employed for renal artery denervation.

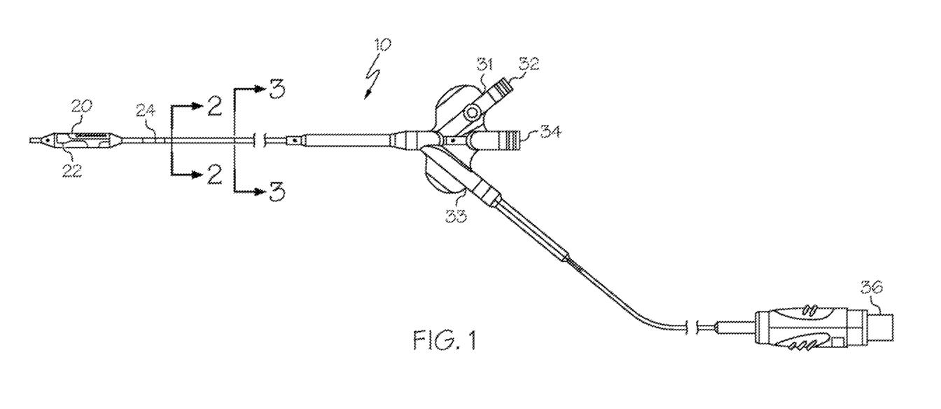

Turning now the drawings, FIG. 1 is a side view of a catheter or catheter assembly 10 having a balloon 20 for renal nerve modulation disposed at the distal end thereof. Catheter 10 includes a manifold 31 having a port 32 for inflation fluid, port 33 wires that run from the electrodes on the flexible circuits 22 to the electric plug 36 to a generator (not shown), and a guidewire lumen 34.

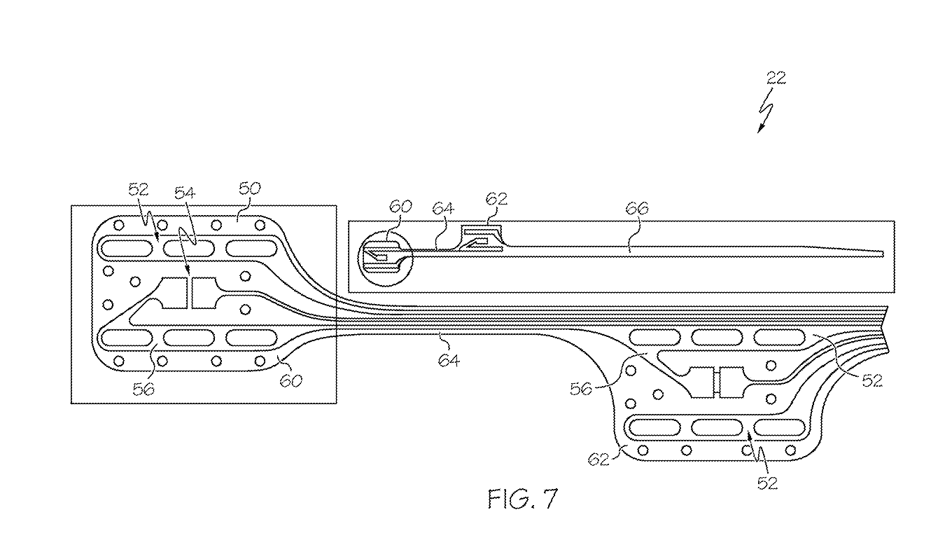

Balloon 20 includes flexible circuits 22 disposed thereon. Balloon 20 is a radially expandable balloon. Balloon 20 is delivered to a treatment site in a patient's vasculature and inflated with fluid supplied through port 32 during use. The balloon is bonded at the distal end to the distal end of an outer catheter shaft 24 and at the proximal end to an inner catheter shaft. Each flexible circuit, explained in more detail with respect to FIG. 6 is formed of a base polymer material 50 comprising two pads 60, 62, each of which contains two pairs of electrodes connected to a power supply at the proximal end of the device via wires disposed within the outer catheter shaft.

The flexible circuit is formed from a relatively rigid polymeric material with copper pathways for conducting current between the electrodes.

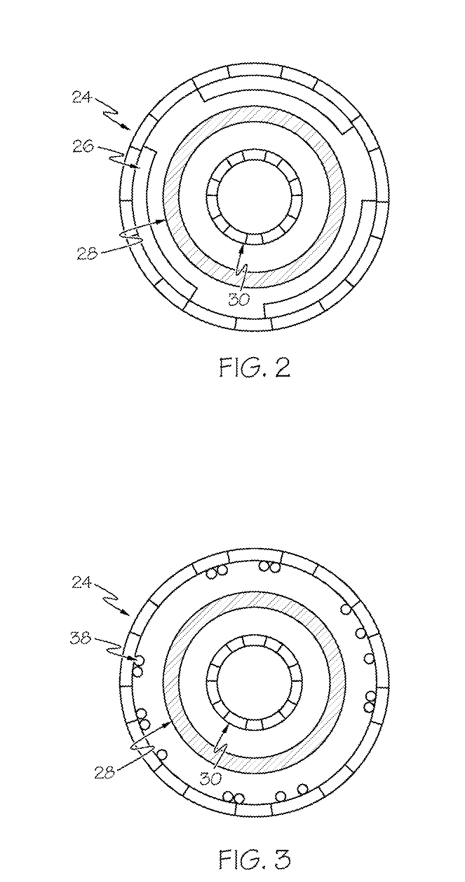

FIG. 2 is a cross-sectional view taken at section 2-2 in FIG. 1 illustrating the outer shaft 24 of catheter 10, electrodes 26 disposed within outer shaft 24, inflation lumen 28 and guidewire lumen 30.

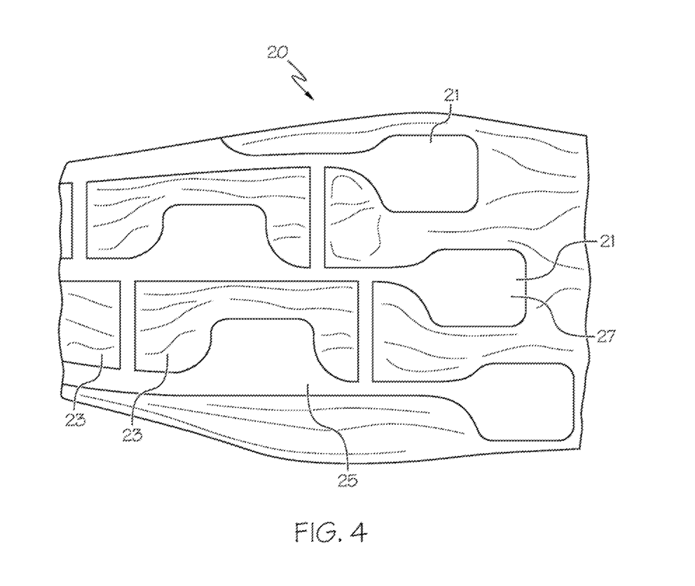

FIG. 3 is a radial cross-sectional view taken at section 3-3 in FIG. 1 further illustrating wires 38 at are distally coupled to electrodes 26. These wires provide power and grounds for the temperature sensors and ablation electrodes.

It has been found that if the flexible circuit 22, typically adhered to the balloon 20 via the use of adhesive, has exposed edges, delamination of the flexible circuit 22 during balloon insertion, refolding and withdrawal from the treatment site.

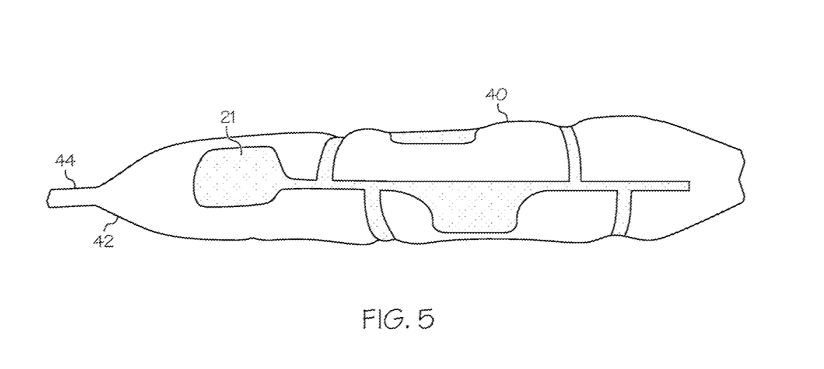

Providing patterned recesses 21 on the outer surface of the balloon 20 which are designed to house and protect the edges of the flexible circuits 22 as shown in FIG. 4 has been found to eliminate catch points and reduce or eliminate any delamination of the flexible circuit 22 from the balloon outer surface.

FIG. 4 is a partial view of a balloon 20 as formed in a static state, i.e. neither inflated nor deflated. In this embodiment, the patterned recesses 21 include a distal region 23, an intermediate 25 and a proximal region 27 which are designed to accommodate a flexible circuit 22 having a distal pad 60 and a proximal pad 62 connected by a distal spline 64. Each pad 60, 62 has an electrode 52 coupled to a thermistor 54, mounted on copper traces for individual temperature control feedback, and a shared ground 56, as shown in FIG. 6.

There is also a proximal spline portion (not shown) that extends from pad 62 to nearly the proximal waist portion of the balloon (see FIG. 1) wherein wires 38 (see FIGS. 2 and 3) are soldered thereto. Wires 38 then run through port 33 to the plug 36 for a generator (not shown in FIG. 1). See also commonly assigned U.S. patent application Ser. No. 14/316,352 and U.S. patent application Ser. No. 61/686,863, each of which is incorporated by reference herein in its entirety.

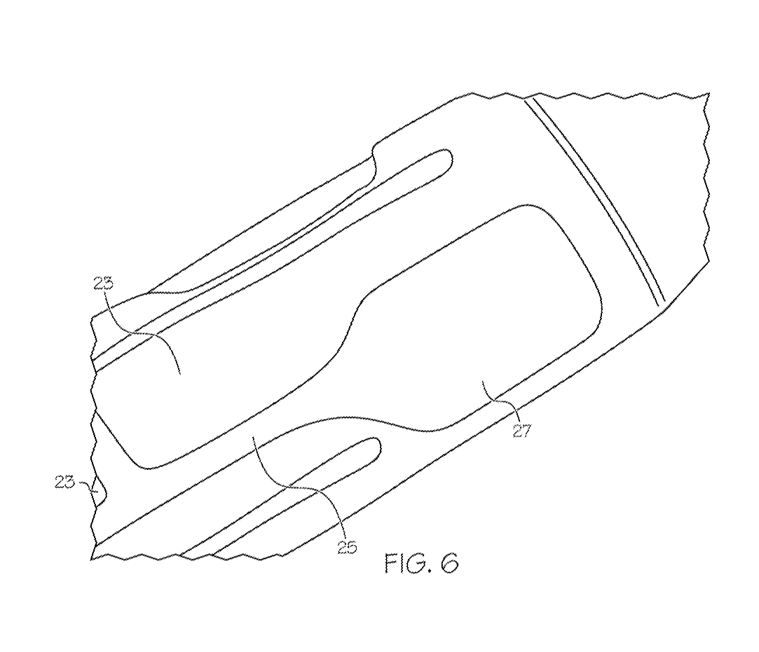

FIG. 5 is a perspective view of an entire balloon similar to that shown in FIG. 4 in an expanded state showing the patterned recesses 21 in the outer surface of the balloon wall.

FIG. 6 is partial enlarged view of a balloon 20 similar to that shown in FIGS. 4 and 5 in an inflated state.

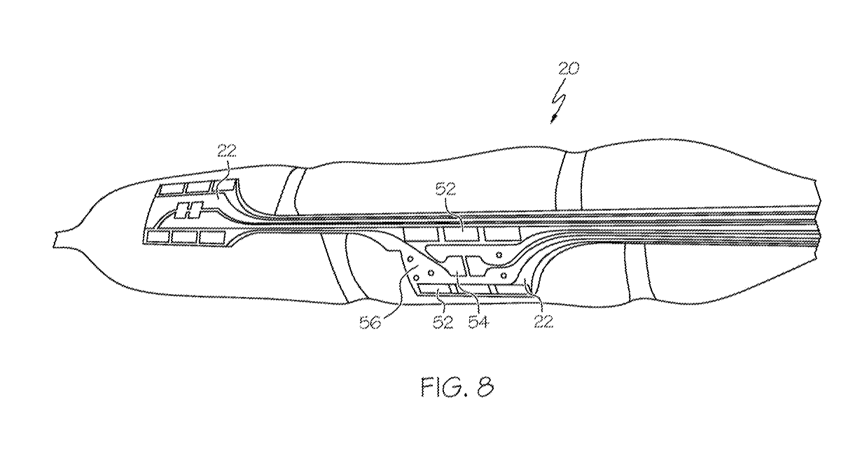

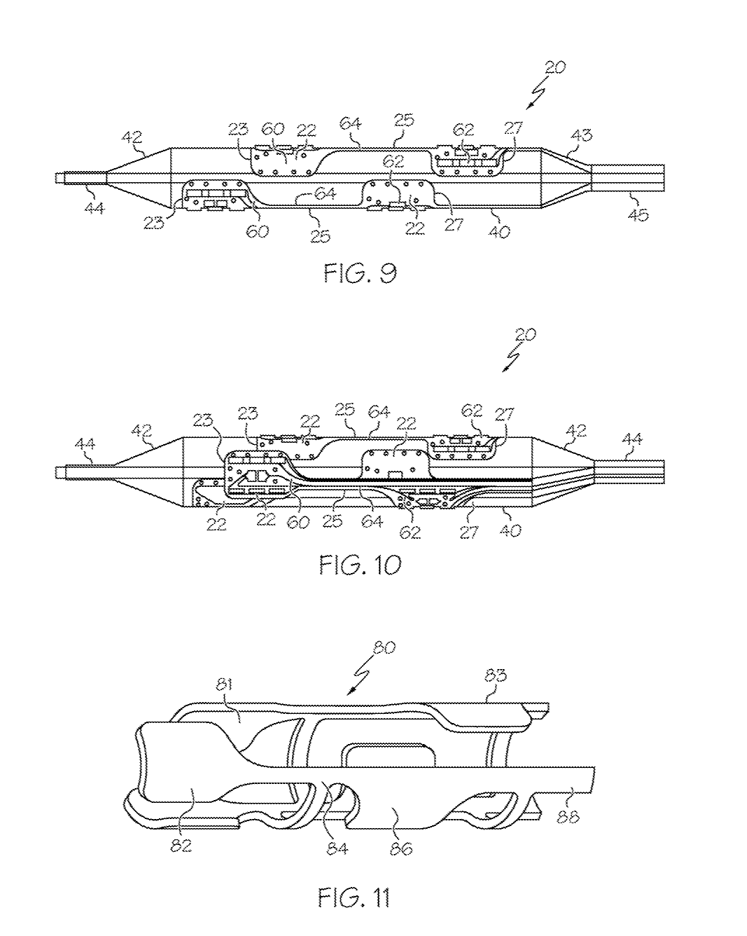

FIG. 7 and FIG. 8 illustrate a balloon 20 having patterned recesses 21 including a distal region 23, an intermediate portion 25 and a proximal portion 27 for accepting pad 60, distal spline 64 and pad 62 respectively.

FIG. 8 is a perspective view of the flexible circuit similar 22 to that shown in FIG. 7 embedded in the patterned recesses (not seen in FIG. 8) of a balloon 20 similar to that shown in FIGS. 5 and 6. Flexible circuit includes electrodes, 52, thermistors 54 and common ground 56.

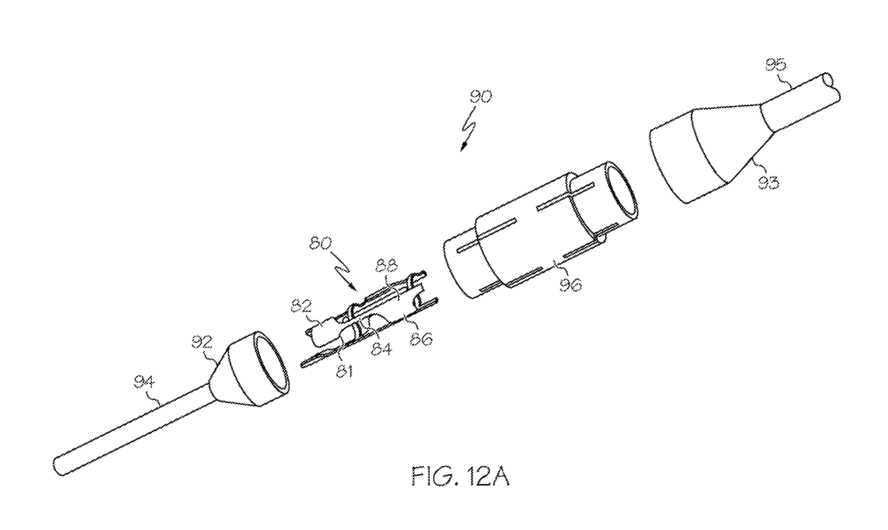

FIG. 9 is illustrative of a balloon that is 4 mm in diameter. Balloon 20 includes a body portion 40, a distal cone portion 42, a distal waist portion 44, a proximal cone portion 43 and a distal proximal waist portion 45 (not shown in FIG. 8).

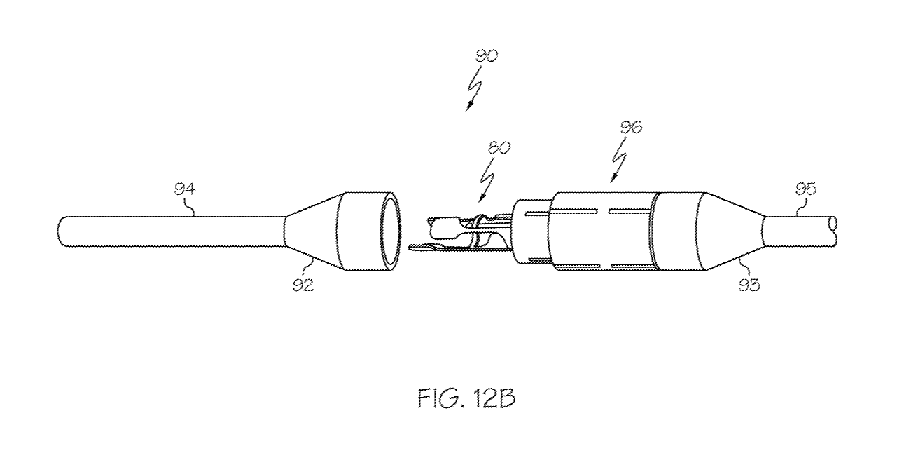

FIG. 10 is a side view of one embodiment of a balloon 20 for renal nerve modulation illustrating four flexible circuits 22 disposed thereon. Alternatively, balloon 20 may have 3, 5 or more flexible circuits disposed thereon. The flexible circuit pocket recesses may extend into the proximal and/or distal cones of the balloon body to provide additional flex circuit edge protection during withdrawal procedures.

Larger balloons of 5, 6, 7 or 8 mm diameter, may include a larger number of flexible circuits such as 3 or more flexible circuits.

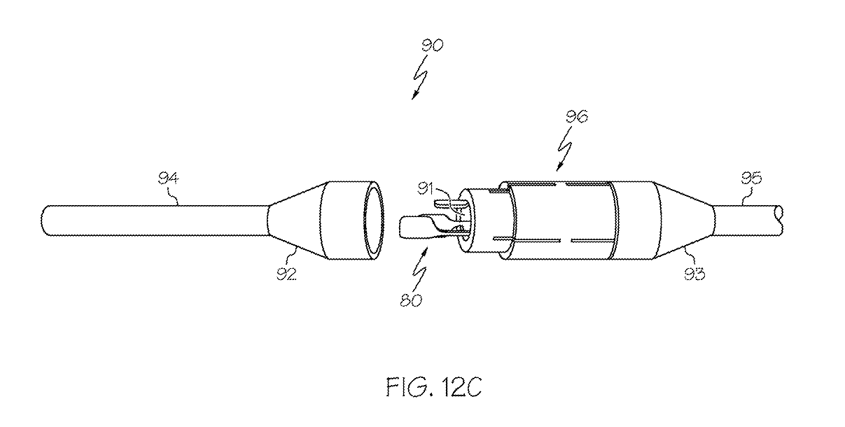

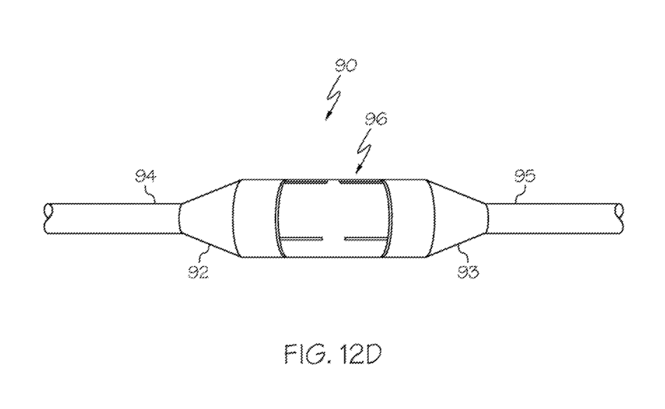

FIGS. 11 and 12A-12B are illustrative of a sleeve 80 and a mold 90 which can be employed in forming the balloon 20 according to the disclosure.

Sleeve 80 is designed for insertion into mold 90. Sleeve 80 has an inner diameter defined by the inner surface 81 of sleeve 80.

As will be explained in more detail below, sleeve 80 includes a distal pad portion 82, an intermediate spline portion 84, proximal pad portion 86 and a proximal spline portion 88 which will form recessed portions 23, 25 and 27 for accepting distal pad 60, spline 64 and proximal pad 62 of flexible circuit 22 respectively.

Sleeve 80 is configured for insertion into mold 90 as shown in FIGS. 12A-12D. The outer diameter of sleeve 80 is defined by the outer surface 83 of sleeve 80 is approximately equal to the inner diameter defined by the inner surface 91 of the body portion 96 of balloon mold 90 (shown in FIG. 12C) which is larger than that of the inner diameter defined by the inner surface 81 of sleeve 80.

Balloon mold 90 further includes a distal cone portion 92, a distal waist portion 94, a proximal cone portion 93 and a proximal waist portion 95.

Sleeve 80 is shown partially inserted in body portion 96 of balloon mold 90 in FIGS. 12B and 12C, and is fully inserted in balloon mold 90 as shown in FIG. 12D. A balloon preform in the form of an extruded tube is inserted in the balloon mold 90 with the sleeve 80 in place and is radially expanded. Conventional balloon molding processes can be employed to form the balloons herein.

For example, the following mold setting parameters were employed for forming a 6 mm PEBAX® 72D recessed balloon using a standard water-based IMS molding station. Minor process changes are made for various diameter balloons and tube lots for optimum production yields.

An extruded tube of Pebax® 72d is prestretched prior to balloon molding. The extruded tube load position was 170 mm and the stretch-to position was 590 mm. Stretching was conducted at ambient temperature at a stretch speed of 200 mm/second. Air pressure inside the tube during prestretching was 0 psi.

The Pebax® 72d balloon version was produced @95° C. water bath temp. The balloon was heat set after formation at 118° C. for 30 seconds.

TABLE 1

| | | | Tension | Hold Time | Travel-time |
|---|---|---|---|---|---|
| StopPoint | Distance | PSI | (grams) | (seconds) | (seconds) |
| 1 | 3400 | 280 | 360 | 3 | 2 |
| 2 | 4600 | 360 | 150 | 6 | 1 |
| 3 | 5200 | 380 | 150 | 10 | 1 |

Distance: Depth mold tool (with extruded tube) is placed into the water hot bath for a given stop-point.
PSI: Air pressure going into the tube that is loaded in the mold tool.
Tension: Amount of force (in grams) on a pneumatic tension arm that pulls vertically on the extruded balloon tube to prevent recoil of the tube during hot bath plunge.
Hold Time: Length of time tool will remain at a given stop-point
Travel-time: Speed rate of travel in Z axis.

The balloon mold may be designed for making any suitable size diameter balloon, depending on its use. For renal nerve modulation, balloon sizes are typically 4-8 mm in diameter.

Smaller balloons may have as few as two flexible circuits and thus sleeve 40 will have two distal pad portions 42, two intermediate or distal spline portions 44 and two proximal pad portions 56.

Larger balloons may have as many as four or more flexible circuits and thus sleeve 40 will incorporate four of more distal pad portions 42, our or more intermediate or distal spline portions 44 and four or more proximal pad portions.

While the above examples are illustrative of the shape of a flexible circuit, other designs are contemplated without departing from the scope of the present disclosure.

Furthermore, the balloons according to the disclosure are not limited to use in renal nerve modulation.

The balloon may be formed of noncompliant polymer materials or semi-compliant or compliant polymer materials.

Compliant balloons are made from relatively soft or flexible polymeric materials. Examples of these materials are thermoplastic polymers, thermoplastic elastomers, polyethylene (high density, low density, intermediate density, linear low density), various copolymers and blends of polyethylene, ionomers, polyesters, polyurethanes, polycarbonates, polyamides, polyvinyl chloride or acrylonitrile-butadiene-styrene copolymers. A suitable copolymer material, polyolefin material is available from E. I. DuPont de Nemours and Co. (Wilmington, Del.), under the trade name Surlyn® Ionomer.

Intermediate compliant balloons are made of polyether-block-amide (PEBA) copolymers and nylon materials.

Non-compliant balloons are made from relatively rigid or stiff polymeric materials. These materials are thermoplastic polymers and thermoset polymeric materials. Some examples of such materials are poly(ethylene terephthalate), polyimide, thermoplastic polyimide, polyamides, polyesters, polycarbonates, polyphenylene sulfides, polypropylene and rigid polyurethanes. Non-Compliant balloons made from poly(ethylene terephthalate) are commonly referred to as PET balloons.

In some embodiments, the balloon is formed of a non-compliant polymer material such as polyethylene terephthalate (PET).

Each flexible circuit is formed from a polymer base material 50 which is typically more rigid than the polymer from which the balloon is formed. In some embodiments, the base of the flexible circuit is formed from Kapton® polyimide available from DuPont™ in Wilmington, Del.

Other suitable polymer materials from which the flexible circuit may be formed include, but are not limited to, polyethylene terephthalate (PET) and polyethylene naphthalate (PEN) available from Dupont Teijin Films in Chester, Va., and a thermoset polyimide may also be employed herein.

An adhesive may be employed to secure each flexible circuit 22 in the patterned recesses 21. The adhesive can be applied in numerous other patterns and shapes without deviating from the scope of the present disclosure.

Any suitable adhesive may be employed providing it is a biocompatible medical grade adhesive including thermoplastic and thermoset adhesives.

In some embodiments, the adhesive is a thermoset adhesive.

In some embodiments, the adhesive is an ultraviolet (UV) curable adhesive.

In one embodiment, the adhesive is a urethane-acrylic adhesive.

One example of a commercially available medical grade urethane-acrylic adhesive is Dymax® 204 CTH available from Dymax® Corporation in Torrington, Conn.

The adhesive may be applied to the balloon, the flexible circuit, or both. Suitably, the adhesive is disposed at least on the portion of the balloon and/or flexible circuit which are in contact with one another.

The flexible circuit 22 and/or the balloon 20 may be textured prior to application of adhesive 70. This results in less delamination of the flexible circuits 22 from the balloon 20. For example, the flexible circuit 22 and/or the balloon 20 can be laser etched prior to application of the adhesive 70. This improves adhesion of the flexible circuit to the balloon. Laser etching of the flexible circuits and/or balloon is disclosed in commonly assigned, copending U.S. patent application Ser. No. 14/316,352, the entire content of which is incorporated by reference herein.

It has been found that electrode attachment robustness can be improved by texturing the outer surface of the balloon and/or the inner surface or bonding surface of the flexible circuit.

The above embodiments are for illustrative purposes only and are not intended to limit the scope of the present disclosure.

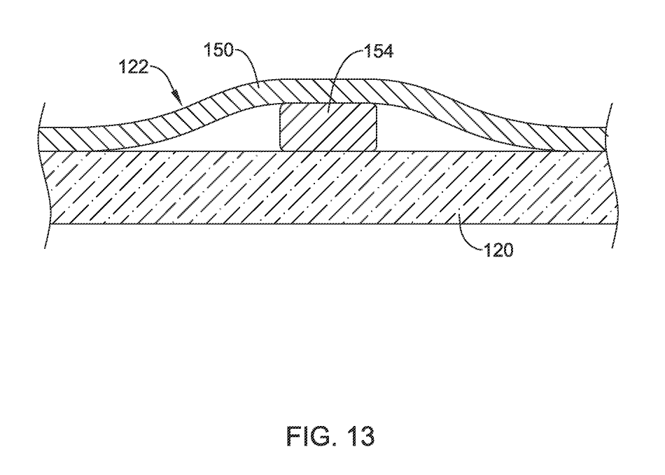

FIG. 13 is a cross-sectional view of an example balloon 120 having a flexible circuit 122 coupled thereto. Flexible circuit 122 may be similar in form and function to other flexible circuits disclosed herein and may include a base material or substrate 150. A temperature sensor 154 may be coupled to substrate 150. Temperatures sensor 154 may take the form of a thermistor, thermocouple, or the like. In this example, temperature sensor 154 projects outward from the surface of substrate 150. Because of this, flexible circuit 122 may protrude outward from the wall of balloon 120 when flexible circuit 122 is coupled to balloon 120. In addition, because of the gap that may be formed adjacent to flexible circuit 122, adhesive used bond flexible circuit 122 to balloon could build up (e.g., and/or “tent”) around temperature sensor 154. These structural aspects could impact the profile of a device and/or impact the foldability (and/or refoldability) of balloon 120.

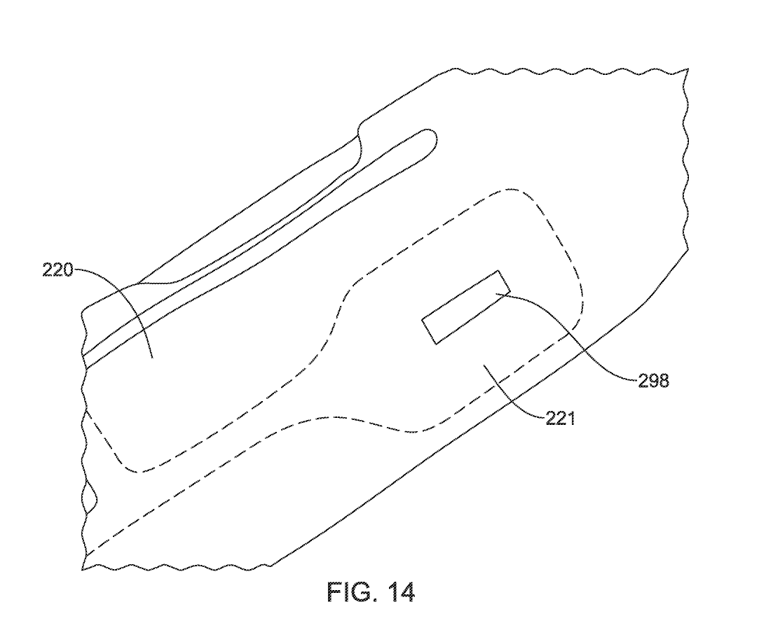

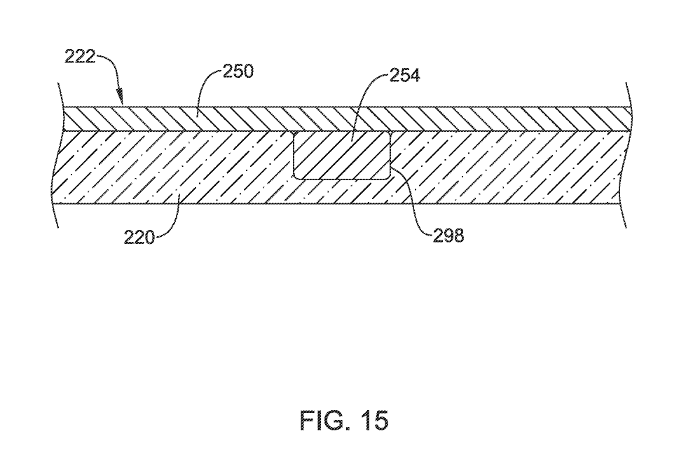

In some instances, it may be desirable to reduce the protrusion of flexible circuit 122 from balloon 122. For example, FIG. 14 illustrates balloon 220 having a temperature sensor recess 298. FIG. 15 illustrate flexible circuit 222 including substrate 250 and temperature sensor 254 extending from substrate 250. When disposed along balloon 220, temperature sensor 254 may fit within temperature sensor recess 298. Temperature sensor recess 298 may be formed by as a pocket in balloon 220, for example during a blow molding or other balloon manufacturing process. For example, an insert may be utilized within a molding tool, by machining the mold (e.g., including a clamshell style or other molds) so as to define recess 298, or the like. Alternatively, temperature sensor recess 298 using an etching or mechanical removing process where a portion of the balloon wall is removed to define temperature sensor recess 298.

Temperature sensor recess 298 may help to reduce the profile of a device, improve foldability/refoldability, and/or reduce the possibility of delamination of flexible circuit 222 from balloon 220. Moreover, temperature sensor recess 298 may allow for more consistent adhesive thicknesses by defining a location where the adhesive can be suitably contained. In addition, temperature sensor recess 298 may aid in manufacturing by serving as a “location marker” that helps to guide flexible circuit 222 into the desired location along balloon 220 as well as provide a mechanical interlocking feature that helps to increase the integrity of the bond between flexible circuit 222 and balloon 220.

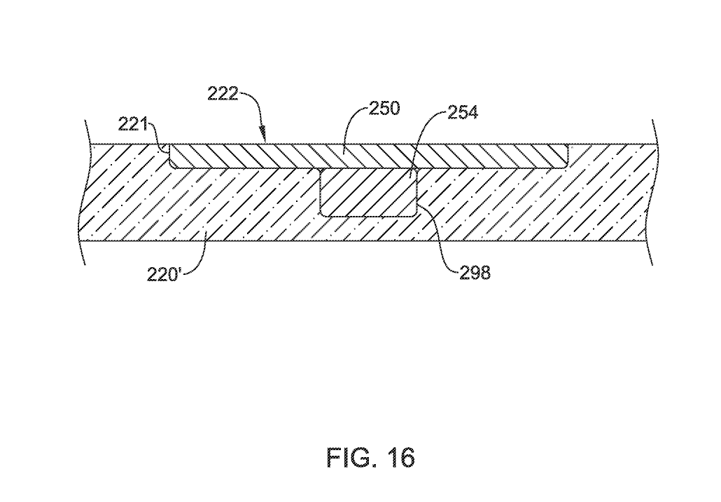

In some of these and in other embodiments, balloon 220 may also include a patterned recess 221 (shown in phantom line in FIG. 14) designed to house a flexible circuit (e.g., in a manner similar to what is disclosed herein). FIG. 16 illustrates balloon 220′ (including both temperature sensor recess 298 and patterned recess 221) where temperature sensor 254 is disposed within temperature sensor recess 298 and where flexible circuit 222 is disposed within patterned recess 221.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

U.S. Patent Application Pub. No. US 2013/0165926 is herein incorporated by reference.

U.S. patent application Ser. No. 14/070,211 is herein incorporated by reference.

U.S. patent application Ser. No. 61/891,257 is herein incorporated by reference.

All published documents, including all US patent documents and US patent publications, mentioned anywhere in this application are hereby expressly incorporated herein by reference in their entirety. Any copending patent applications, mentioned anywhere in this application are also hereby expressly incorporated herein by reference in their entirety. Citation or discussion of a reference herein shall not be construed as an admission that such is prior art.

What is claimed is:

1. A medical balloon, comprising:
a balloon wall formed from a polymeric material, the balloon wall having an inner surface and an outer surface;
the balloon wall comprising patterned recesses in the outer surface thereof; and
flexible circuits disposed within the patterned recesses, the flexible circuits are defined by an outer perimeter.

2. The medical balloon of claim 1, wherein the balloon is a renal denervation balloon.

3. The medical balloon of claim 1, wherein the patterned recesses reflect the outer perimeter of the flexible circuits.

4. The medical balloon of claim 1, wherein the flexible circuits comprise two pads connected by a distal spline.

5. The medical balloon of claim 1, wherein the balloon comprises 2 to 4 patterned recesses and 2 to 4 flexible circuits, one circuit disposed in each of said 2 to 4 patterned recesses.

6. The medical balloon of claim 1, wherein the balloon wall comprises a body, waist and cone portions, the balloon wall at the patterned recesses is the same thickness as a remainder of the body of the balloon.

7. The medical balloon of claim 1, wherein the flexible circuits are disposed within the recesses such that they are flush or less than flush with a remainder of the balloon wall.

8. The balloon of claim 1, wherein the polymer material forming the balloon wall is a non-compliant polymer material.

9. The balloon of claim 8, wherein the polymer material forming the balloon wall is polyethylene terephthalate.

10. The balloon of claim 1, wherein the flexible circuit is a composite material that is more rigid that the polymer material forming the balloon wall.

11. The balloon of claim 1, wherein the base of the flexible circuit is polyimide.

12. The balloon of claim 1, wherein the flexible circuits are adhered to the balloon outer surface with an adhesive.

13. The balloon of claim 1, wherein at least some of the flexible circuits include a temperature sensor.

14. The balloon of claim 13, wherein a temperature sensor recess is formed in the balloon wall and wherein the temperature sensor is disposed within the temperature sensor recess.

15. The balloon of claim 14, wherein the temperature sensor recess is disposed along at least some of the patterned recesses.

16. A method of forming a medical balloon defined by a shape, the medical balloon having flexible circuits defined by an outer perimeter disposed on an outer surface of the balloon, the method comprising:
providing a balloon mold in the shape of the balloon, the balloon mold comprising a first inner surface diameter;
providing an inner sleeve within the balloon mold that defines the perimeter of the flexible circuits, the inner sleeve comprising a second inner surface diameter that is less than the diameter of the first inner surface;
providing a balloon preform; and
radially expanding the balloon preform in the balloon mold to form a medical balloon;
wherein the inner sleeve defines patterned recesses in the outer surface of the medical balloon.

17. A mold for forming an expandable medical balloon configured to accept flexible circuits on an outer surface of the balloon, the flexible circuits defined by a perimeter, the mold comprising:
an outer shell defining the shape of the balloon, the outer shell comprising a body portion, and waist and cone portions, the body portion of the outer shell defined by a length and having an inner surface comprising a first diameter;
an inner sleeve defining the perimeter of the flexible circuits, the inner sleeve having an inner surface comprising a second diameter, the second diameter is less than the first, the inner sleeve extends no further than the length of the body portion of the mold, the inner sleeve is insertable in the outer shell.

18. The mold of claim 17, wherein the inner sleeve comprises a distal pad portion and a proximal pad portion connected by a spline portion which defines the shape of a flexible circuit.

19. The mold of claim 17, wherein the inner sleeve comprises at least two distal pad portions, at least two proximal pad portions and at least two spline portions.

20. The mold of claim 17, wherein the inner sleeve comprises at least four distal pad portions, at least four proximal pad portions and at least four spline portions.
